(19) United States
(12) Patent Application Publication
(10) Pub. No.: US 2023/0408418 A1
(43) Pub. Date: Dec. 21, 2023

(54) Multi-Modal Diagnostic Test Apparatus

(71) Applicant: Axxin Pty Ltd, Fairfield, Victoria (AU)

(72) Inventors: William Robb Hopper, Fairfield, Victoria (AU); Daniel Murawczuk, Fairfield, Victoria (AU); Anthony Stephen Lawlor, Fairfield, Victoria (AU); Gavin Ramanathan, Fairfield, Victoria (AU); Anthony Do, Fairfield, Victoria (AU)

(21) Appl. No.: 18/031,853

(22) PCT Filed: Oct. 14, 2021
(86) PCT No.: PCT/AU2021/051200

§ 371 Date: Apr. 13, 2023

(30) Foreign Application Priority Data
Oct. 14, 2020 (AU) 2020903729

(51) Int. Cl. G01N 21/84; G01N 21/78; G01N 33/543

(52) U.S. Cl. CPC G01N 21/8483; G01N 2021/0112; G01N 33/54388; G01N 21/78

(57) ABSTRACT

A multi-modal diagnostic test reading apparatus, comprising:
a diagnostic test assembly receiving component;
at least one image sensor;
a plurality of light sources having respective different spectral properties; and
a controller;
wherein the controller is configured to:
(i) control operation of the light sources and the at least one image sensor to acquire a plurality of images, each of the acquired images representing at least a corresponding portion of the diagnostic test assembly as illuminated by a corresponding one of the light sources; and
(ii) process the acquired images to determine a diagnostic test result of the diagnostic test, the diagnostic test result being dependent upon the processed images representing illumination by respective ones of the light sources having respective different spectral properties.

### TECHNICAL FIELD

This invention relates to the general fields of diagnostic and biomedical testing using fluorescent and/or colorimetric assays. More particularly, the invention relates to a multi-modal test instrument or apparatus for reading lateral flow strips or fluidic cartridges, suitable for use in diagnostics, including Point-of-Care (POC) medical testing.

### BACKGROUND

Reference to any prior art in the present specification is not an acknowledgement or suggestion that this prior art forms part of the common general knowledge in any jurisdiction or that this prior art could reasonably be expected to be understood, regarded as relevant and/or combined with any other pieces of prior art by a skilled person in the art.

Lateral Flow Strips

Immunochromatography or lateral flow strips, herein referred to as lateral flow strips, are commonly used in rapid diagnostic applications.

Typical components of a lateral flow strip include an absorptive sample application or input pad at a first end of the strip, a membrane along which the analyte flows, a conjugate pad between the input pad and membrane containing, for example, dried bio-active conjugates, and a waste adsorbing pad at the opposite end of the strip. The components are bonded by an adhesive layer onto a carrier strip or cartridge, which is typically plastic.

One or several test regions, comprising, for example, test lines or multi-dot arrays, are immobilised on the lateral flow strip membrane, and contain capture antigens or antibodies for the target or targets of interest. Further, the membrane typically includes a control region containing affinity ligands to indicate whether or not the biomolecules from the conjugate pad have migrated along the strip during the test run.

Lateral flow and other similar types of biomedical test strips are widely used to qualitatively diagnose a range of medical conditions from pregnancy to infectious diseases, for example influenza, by determining the presence or absence of some pathogen or biomarker in a sample collected from a subject. These tests often involve colorimetric immunoassays designed to produce control and test lines or other shaped regions that are visible to a human user.

Colorimetric lateral flow strips with visible target and control regions are often contained in a plastic cartridge having an opening for sample introduction, and an open or transparent “window” for viewing the test regions. For example, the test result of a pregnancy test strip can be viewed in the home by simple visual inspection under natural or other ordinary ambient lighting conditions. Semi-quantitative results are possible with colorimetric strips, wherein the visual intensity or obviousness of an immunoassay capture region is indicative of the quantity of the target within the sample.

Lateral flow strips can also be used in quantitative analyses. For example, a fluorescent label can be added to either antigens or antibodies at an immunoassay capture region of a lateral flow strip, such that the detected intensity of a fluorescent signal produced at the immunoassay capture region is proportional to the amount of target analyte present in the sample. Quantification can also be performed with colorimetric tests; for example, by using absorption/reflection measurements of incident light at a capture region of the strip.

### Fluidic Sample Test Cartridges

Other known investigative procedures involve the use of fluidic biological or environmental sample test cartridges, chips or slides, herein referred to as fluidic cartridges.

A fluidic cartridge can be constructed to operate in a similar way to a lateral flow strip, in that sample fluid flows laterally through a cartridge and into one or more reaction chambers. The cartridge may include optical areas or immunoassay binding at features within the cartridge, which may be optically detected within a viewing area or viewing window.

Typical components of a fluidic cartridge include a sample input port which connects directly or via a fluidic channel to a reaction chamber. The cartridge may also include a vent and vent membrane to remove air from the reaction chamber.

A test involving a fluidic cartridge typically involves preloading a reaction chamber with a soluble, dried or lyophilised reagent, and subsequently introducing a fluid sample into the reaction chamber. Depending on the specific test, a biological fluid sample may be, for example, blood, urine, saliva, plasma, semen, sputum, breast milk, or cerebrospinal fluid. A fluid environmental sample may be, for example, water from a lake, reservoir, aquifer, or stream.

A fluidic cartridge may be relatively simple, wherein a single sample is introduced into one preloaded reaction chamber, or more complex, including, for example, multiple sample input ports, mixing wells, fluidic channels, reaction chambers, etc. for performing more complicated sample preparation steps and/or multiplexed tests. Further, a fluidic cartridge may include only a single layer wherein movement of fluid within the cartridge occurs in a single plane, or multiple layers where fluid can also move between layers. In multiple layer cartridges, the layers may or may not move relative to each other to assist fluid flow throughout the cartridge.

Pursuant to lateral flow assays, fluidic cartridge assays may involve colorimetry and/or the use of fluorescence. For example, in a visual colorimetric test, the colour of the liquid within the reaction chamber may change, depending on the presence or absence of some target analyte in a sample.

Fluorescence-based tests using either fluidic cartridges or lateral flow strips require a stimulating signal to stimulate the emission of a second signal (from a reaction chamber or test area) that is indicative of the test result. The stimulating signal may be an optical signal of a specific wavelength or wavelength range.

Typically, the stimulating/excitation signal will consist of a narrow band of wavelengths, which can be produced using a bandpass filter in combination with a broadband signal source, or simply by using a narrowband signal source. The stimulated emission signal is then typically detected through a second filter that excludes the wavelengths of the stimulating signal, such that only the stimulated emission signal is detected by the sensor.

### Diagnostic Test Readers

The use of diagnostic test readers can provide a significant improvement in the reliability, repeatability and sensitivity of tests using either fluidic test cartridges or lateral flow-strips, even where a test has visually readable results.

Human-read visual test results are susceptible to interpretation errors, and different people can have different levels of visual acuity and proficiency at interpreting test results. For example, where a colorimetric lateral flow strip is used to test for the presence or absence of some target analyte, a false negative may result from a faint or barely visible (low chromaticity) test line.

A variety of readers are known. Typically, these fall into two categories, the first being readers that read colorimetric tests, where optical detection may involve absorption or reflection methods, and the second being readers that read tests with fluorescent signal outputs. There are, however, a number of functional limitations to both reader types.

For example, colorimetric readers have a high detection sensitivity when using a narrowband illumination spectrum with a corresponding narrowband image sensor. However, some lateral flow strips that test for more than one target often have immunoassay capture regions with different absorption spectra, e.g., different colours, for each test. Similarly, multiplexed colorimetric fluidic cartridges may require the interpretation of different colours. Often in this case, a reader with wide bandwidth illumination such as white light, along with a colour image sensor, is used. However, this approach does not provide the same level of detection sensitivity as narrowband illumination and detection. There is therefore a need for improved colorimetric readers, capable of more accurately reading test results having multiple spectral properties.

Further, many fluorescence-based readers require the use of a spectrometer within a laboratory setting. Some point-of-care fluorescent readers use UV light to analyse blood samples. However, known point-of-care fluorescence readers are less accurate than spectrometer-based readers. There is therefore a need for point-of-care fluorescence-based diagnostic test readers with improved accuracy.

It is desired to alleviate or overcome one or more difficulties of the prior art, or to at least provide a useful alternative.

### SUMMARY

In work leading up to the invention, the inventors determined that combining colorimetric and fluorescence-based readings enables broader and more accurate test results. Moreover, there are specific applications in which combined test results are beneficial.

Accordingly, embodiments of the present invention include readers with multiple reading modes; for example, a reader with separate reading modes for reading colorimetric tests with different spectral properties, and/or further reading modes for reading fluorescence-based tests. The reader may include further reading modes for reading cartridge features and other useful visual features. Measurements in these separate reading modes are combined to obtain a final test result.

In accordance with some embodiments of the present invention, there is provided a multi-modal diagnostic test reading apparatus, comprising:
a diagnostic test assembly receiving component;
at least one image sensor;
a plurality of light sources having respective different spectral properties; and
a controller;
wherein the controller is configured to:
(i) control operation of the light sources and the at least one image sensor to acquire a plurality of images, each of the acquired images representing at least a corresponding portion of the diagnostic test assembly as illuminated by a corresponding one of the light sources; and
(ii) process the acquired images to determine a diagnostic test result of the diagnostic test, the diagnostic test result being dependent upon the processed images representing illumination by respective ones of the light sources having respective different spectral properties.

In some embodiments, the apparatus further comprises an optical filtering component operable by the controller to selectably locate a corresponding optical filter of one or more optical filters between the image sensor and the diagnostic test assembly to filter corresponding wavelengths from the image sensor when acquiring one or more corresponding images of the acquired images.

In some embodiments, the controller is configured to include an operating mode wherein the plurality of images include:
an absorption/reflection-based image of a first colorimetric signal produced at a first test region of the diagnostic test assembly; and
an absorption/reflection-based image of a second colorimetric signal produced at a second test region of the diagnostic test assembly;
wherein the spectral properties of the first colorimetric signal are different to the spectral properties of the second colorimetric signal.

In some embodiments, the image sensor includes a Bayer filter, and while acquiring respective images of the plurality of images, the controller is configured to selectively use only respective different subsets of pixels of the image sensor selected from three subsets of pixels of the image sensor with red, blue and green Bayer filter elements, respectively.

In some embodiments, the controller is configured to include an operating mode wherein the plurality of images include:
an absorption/reflection-based image of a colorimetric signal produced at a first test region of the diagnostic test assembly; and
a fluorescence-based image of a fluorescent signal produced at a second test region of the diagnostic test assembly.

In some embodiments:
the colorimetric signal is a signal produced by colloidal gold labelled particles; and
the fluorescent signal is a signal produced by europium chelate fluorescence labelled particles.

In some embodiments, the first and second test regions are first and second immunoassay capture lines of a lateral flow strip.

In some embodiments, the controller is configured to include an operating mode wherein the plurality of images include:
an absorption/reflection-based image of a modified sample contained within a diagnostic test assembly, relating to a first property of the sample; and
multiple fluorescence-based images of a fluorescent signal produced by the modified sample relating to a second property of the sample.

In some embodiments:
the modified sample is blood mixed with a buffer solution;
the first sample property is an amount of haemoglobin in the sample;
the second sample property is an amount of nicotinamide adenine dinucleotide phosphate hydrogen (NADPH) produced while running the test;
wherein the controller is configured to determine, based on changes in the multiple fluorescence-based images over time, an amount of glucose-6-phosphate dehydrogenase (G6PD) present in the sample; and
wherein determining the test result comprises calculating the amount of G6PD relative to the amount of haemoglobin in the sample.

In some embodiments, the controller is configured to include an operating mode wherein the plurality of images include:
a first, absorption/reflection-based image of one or more visual features within a test area of the diagnostic test assembly; and
a second image of at least a signal produced at a test region of the diagnostic test assembly, the second image being an image of either:
   a fluorescent signal; or
   a colorimetric signal;
      wherein the diagnostic test result is dependent upon the one or more visual features and the signal of the processed images.

In some embodiments, the one or more visual features comprise one or more of:
flow of a coloured sample through a fluidic test cartridge;
flow of a coloured sample along a lateral flow strip;
wetting of a lateral flow strip due to flow of a transparent sample;
variation in illumination;
background staining on a lateral flow strip;
dirt, dust, or imperfections of a lateral flow strip; and
reflections from a reflective surface of the viewing window.

In some embodiments:
the one or more visual features comprise background staining and variation in illumination level; and
the controller is configured to subtract the first image from the second image to produce a third image with reduced contribution from the background staining or variation in illumination level,
wherein the third image is used to determine the diagnostic test result.

In some embodiments, the controller is configured to include an operating mode wherein the plurality of images include one or more absorption/reflection-based images of one or more features of the diagnostic test assembly, the diagnostic test result being dependent upon the one or more visual features, and wherein the one or more features comprise one or more of:
the outline of the diagnostic test assembly;
a viewing window of the diagnostic test assembly;
a data code printed or etched on the diagnostic test assembly; and
a label of or affixed to the diagnostic test assembly.

In some embodiments:
the one or more features comprise the data code,
the controller is configured to obtain information from the data code; and
the controller processes the information obtained from the data code to determine parameters including a test identifier, wherein one or more of the parameters are used to determine the diagnostic test result, and are displayed together with the diagnostic test result.

In some embodiments, the apparatus further comprises one or more optical diffusers positioned between one or more of the light sources and a diagnostic test assembly received in the apparatus to improve illumination of the diagnostic test assembly.

In some embodiments, the controller is configured to automatically determine one or more operating modes for acquiring the plurality of images, and to control operation of the light sources and the at least one image sensor in accordance with the determined one or more operating modes to acquire the plurality of images.

In some embodiments, the controller is configured:
(a) to control operation of the light sources and the at least one image sensor to acquire at least one image of the plurality of images;
(b) to process the at least one image to determine one or more operating modes for acquiring one or more other images of the plurality of images; and
(c) to control operation of the light sources and the at least one image sensor in accordance with the determined one or more operating modes to acquire the one or more other images of the plurality of images.

In some embodiments, the at least one image includes at least one absorption/reflection-based image of one or more of:
an outline of the diagnostic test assembly;
a data code printed on or etched into the diagnostic test assembly; and
a label of or affixed to the diagnostic test assembly;
wherein the controller is configured to determine an operating mode for acquiring at least one of the one or more other image by processing the at least one image to determine at least one of:
a type of the diagnostic test assembly represented in the image; and
a type of the diagnostic test of the diagnostic test assembly.

In accordance with some embodiments of the present invention, there is provided a process executed by at least one processor of a multi-modal diagnostic test reading apparatus comprising at least one image sensor and a plurality of light sources having respective different spectral properties, the process comprising the steps of:
(i) controlling operation of the light sources and the at least one image sensor to acquire a plurality of images, each of the acquired images representing at least a corresponding portion of the diagnostic test assembly as illuminated by a corresponding one of the light sources; and
(ii) processing the acquired images to determine a diagnostic test result of the diagnostic test, the diagnostic test result being dependent upon the processed images representing illumination by respective ones of the light sources having respective different spectral properties.

In some embodiments, the process further comprises a step of automatically determining one or more operating modes for acquiring the plurality of images; wherein the step of controlling comprises controlling operation of the light sources and the at least one image sensor in accordance with the determined one or more operating modes to acquire the plurality of images.

In some embodiments, the step of controlling comprises:
(a) controlling operation of the light sources and the at least one image sensor to acquire at least one image of the plurality of images;
(b) processing the at least one image to determine one or more operating modes for acquiring one or more other images of the plurality of images; and
(c) controlling operation of the light sources and the at least one image sensor in accordance with the determined one or more operating modes to acquire the one or more other images of the plurality of images.

In accordance with some embodiments of the present invention, there is provided at least one computer-readable storage medium having stored thereon processor-executable instructions and/or FPGA configuration data that, when executed by at least one processor of a multi-modal diagnostic test reading apparatus, and/or when used to configure an FPGA of a multi-modal diagnostic test reading apparatus, cause the at least one processor and/or the FPGA to execute any one of the above processes.

### BRIEF DESCRIPTION OF THE DRAWINGS

Some embodiments of the present invention are hereinafter described, by way of example only, with reference to the accompanying drawings, wherein:

FIG. 1A is a schematic illustration showing typical components of a prior art lateral flow strip, with immunoassay capture lines present in a test area of the strip;

FIG. 1B is a schematic plan view of a portion of a prior art cartridge configured to house a lateral flow strip;

FIG. 2A is a first view of a prior art fluidic cartridge;

FIG. 2B is a second view of a prior art fluidic cartridge;

FIG. 3A is a first view of a multi-modal reader according to an embodiment of the present invention, a cartridge containing a lateral flow strip being depicted inside a cartridge drawer of the reader;

FIG. 3B provides a non-limiting second view of a multi-modal reader according to an embodiment of the present invention.

FIG. 3C provides a non-limiting view of a multi-modal reader according to another embodiment of the present invention, with an opening for receiving a cartridge, rather than a drawer.

FIG. 3D provides a non-limiting view of a multi-modal reader according to an embodiment of the present invention, with an opening for receiving a cartridge. By way of example only, the reader is depicted with a fluidic cartridge.

FIG. 4 illustrates the interaction between certain components of an example embodiment of the present invention.

FIG. 5 provides a block diagram of a control system of an example embodiment of the present invention.

FIG. 6 provides an approximate set of illumination emission wavelength curves for ultraviolet, blue, green and red LEDs.

FIG. 7 provides an example of a known Bayer filter arrangement, comprising an array of green, red and blue filters.

FIG. 8 depicts an example dual-mode LED array for illuminating or stimulating at least the test area of a lateral flow strip or fluidic cartridge.

FIG. 9 depicts a lateral flow strip, and captured images of a test region of the lateral flow strip, wherein the images are captured in two different reading modes of a multi-modal reader. A depiction of a third, calculated image is also provided.

FIG. 10 provides an example NADPH fluorescence versus time plot used in a test for G6PD, wherein the slope of the graph can be used to estimate the amount of G6PD present in a sample.

FIG. 11 depicts two images of a cartridge, wherein the first represents an image captured using a feature reading mode of a multi-modal device, and the second represents an image captured in a fluorescence reading mode.

FIG. 12 is a flow diagram of a multi-modal diagnostic test process in accordance with some embodiments of the present invention.

### DETAILED DESCRIPTION

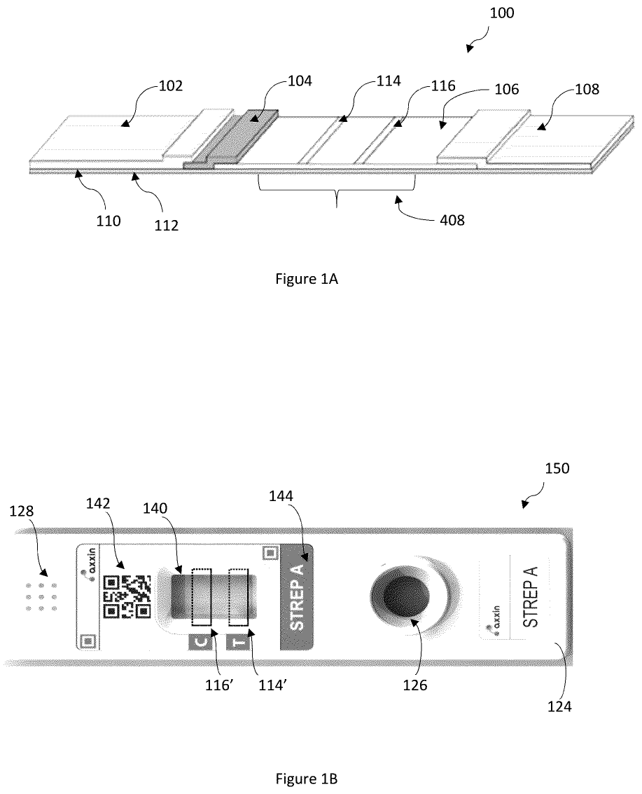

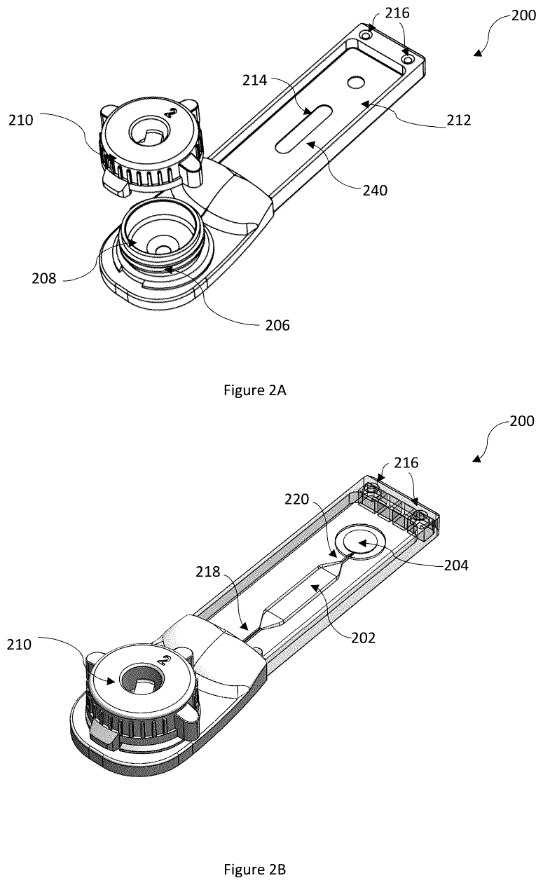

FIG. 1 shows a typical prior art lateral flow strip 100 as commonly used in rapid diagnostic applications. The strip 100 includes an absorptive sample application or input pad 102, a conjugate pad 104, a membrane 106 along which the analyte flows, and a waste adsorbing pad 108. These components are bonded by an adhesive layer 110 onto a carrier strip 112, usually constructed from plastic sheet.

Immobilised on the typically nitrocellulose membrane are one or more test regions or test lines 114, containing capture antigens or antibodies for the target or targets of interest. Control region or line 116 contains control capture antigens or antibodies. As described above, visible, coloured or fluorescent labels are incorporated, such that the test result is displayed as one or more visible or otherwise optically detectible lines at the test line(s) 114 and/or the control line 116 of a test area 408. Commonly used coloured particles include latex, which produces a blue colour, and gold, which produces a red colour. Other lateral flow strip and cartridge arrangements are known, including, for example, lateral flow strips with dots or multi-dot arrays instead of lines.

FIG. 1B shows an example prior art immunoassay cartridge or cassette 150. The cartridge 150 may be configured to house a lateral flow strip, for example the lateral flow strip 100 of FIG. 1. Cartridge 150 comprises a plastic housing 124 which may be, for example, constructed of injection moulded plastic. Here, the cartridge further includes a sample input port 126 through which a sample is added to the lateral flow strip 100, a viewing window 140, a unique quick response (QR) code 142, a label 144 and waste pad ventilation holes 128. Regions 114′ and 116′ of the viewing window 140 provide visibility of the test and control lines 114, 116 of the lateral flow strip 100, respectively.

FIGS. 2A and 2B provide two views of an example prior art biological sample test cartridge 200. FIG. 2B shows the cartridge 200 with its cover plate/panel 214 removed to show components obscured by the cover plate/panel 214 in FIG. 2A. The cartridge 200 includes a sample chamber 208 into which a sample is loaded, a cap 210, a viewing window 240 which provides a view to a reaction chamber 202, a cover plate/panel 214, a ventilation hole 212 which provides an outlet for ventilation membrane 204, attachment portions 216, and a seal 206.

Turning now to FIG. 2B, in this second view, the cap 210 is screwed in place. The cap 210 may include a protruding piercing portion (not shown), such that, in operation, closing the cap 210 pierces the seal 206. The seal may comprise, for example, a thin layer of foil.

Piercing the seal 206 allows the sample, which may be mixed with a buffer solution, to move from the sample chamber 208 to the reaction chamber via a fluidic channel 218. The reaction chamber 202 may be preloaded with a soluble, dried or lyophilised reagent, and is where the test then takes place.

In FIG. 2B, the cover 214 is removed to show the fluidic channel 218 between the sample chamber 208 and reaction chamber 202, and a further fluidic channel 220 between the reaction chamber 202 and the ventilation membrane 204. This second channel 220 allows air to escape from the reaction chamber 202. The depicted fluidic cartridge 200 comprises a single reaction chamber 202, however some prior art cartridges comprise multiple reaction chambers for performing multiplexed testing.

Both lateral flow strip and fluidic cartridge-based tests may involve colorimetric and/or fluorescence-based tests. Fluorescence-based tests require an excitation source for the test results to be detectable by a reader.

An illumination source of a specific wavelength or wavelength range may also be used for improved or targeted absorption/reflection-based readings, herein referred to as AR-based readings. In AR-based readings, an incident light source is used to illuminate a lateral flow strip 100, a cartridge 150, 200, or some portion of a strip or cartridge, for example a viewing window 140, 240 or test area 408. Depending on the surface properties of each point the incident illumination hits, specific wavelengths are reflected, transmitted or absorbed. The wavelengths of the reflected light (i.e., the wavelengths that are not transmitted or absorbed) are detected by a sensor, and may be used to determine, for example, properties of the area being read. Alternatively, relative differences in detected wavelengths may be used to detect, for example, edges of a cartridge feature.

Example excitation/illumination sources include broadband mercury-arc or tungsten halogen lamps, laser sources, for example UV lasers, compact violet 405 nm lasers, 488 nm blue-green argon lasers, 543 nm helium-neon green lasers, 633 nm helium-neon red lasers or mixed gas lasers e.g. krypton-argon lasers, or one or more light emitting diodes (LEDs). LEDs and laser diode components have the advantages of being compact, solid-state, lower in cost and energy consumption, and longer lifetimes. These components are therefore suited to point-of-care/portable devices. Further, like lasers, single colour LEDs and laser diodes emit wavelengths within a narrow range, making them suitable for either direct use in the instrument, or use with a bandpass filter.

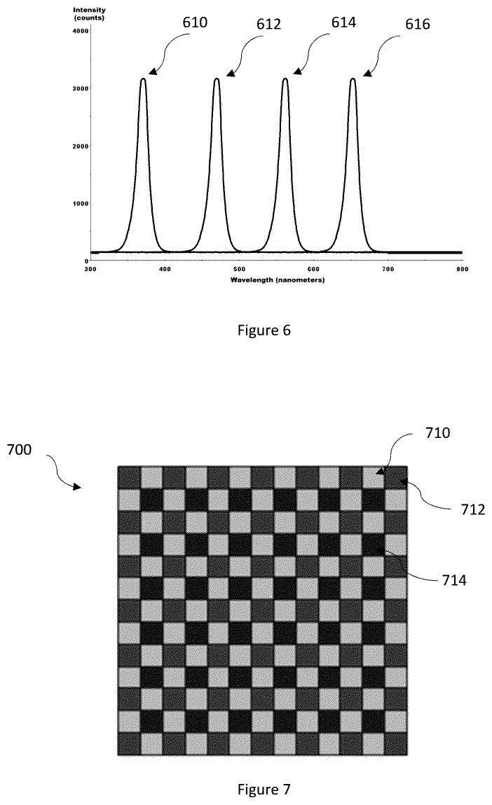

FIG. 6 is a graph showing an approximate set of emission wavelength curves for ultraviolet LEDs 610, blue LEDs 612, green LEDs 614 and red LEDs 616, which may be used in some embodiments of the present invention, however other wavelength LEDs are also available, and may also be used in some embodiments of the present invention. As will be described in detail below, the wavelength of the illumination or excitation source is selected based on specific test reading requirements.

As described below, embodiments of the present invention include a multi-modal diagnostic test reading apparatus and a multi-modal diagnostic test reading process that determine a diagnostic test result from a diagnostic test assembly (such as a lateral flow strip cartridge or fluidic cartridge) by illuminating the diagnostic test assembly (or portion(s) thereof) using multiple light sources having respective different spectral properties, and where determination of the diagnostic test result relies on those different spectral properties.

The phrase “diagnostic test result” as used in this specification is to be construed broadly to include information relevant for its assessment. For example, where a diagnostic test result provides a binary output representing a determination as to whether a subject does or does not have a particular disease or other condition or characteristic, the diagnostic test result can include an indication of the accuracy or reliability of that determination. Similarly, a diagnostic test result can include or be in the form of an indication that the determination (or measurement) could not be made.

As described below, in some embodiments the multi-modal diagnostic test reading apparatus automatically determines one or more operating modes from a received diagnostic test assembly, and then controls the operation of its light sources, image sensor(s) and (if present) optical filter(s) in accordance with the determined operating mode(s) to acquire corresponding images of the diagnostic test assembly or portion(s) thereof under different spectral illuminations. The diagnostic test result is then determined from those images.

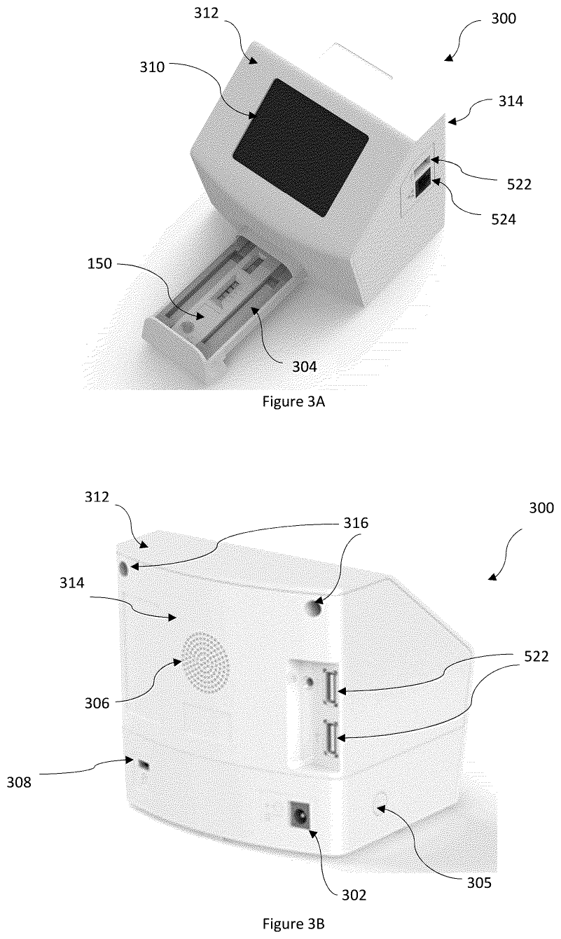

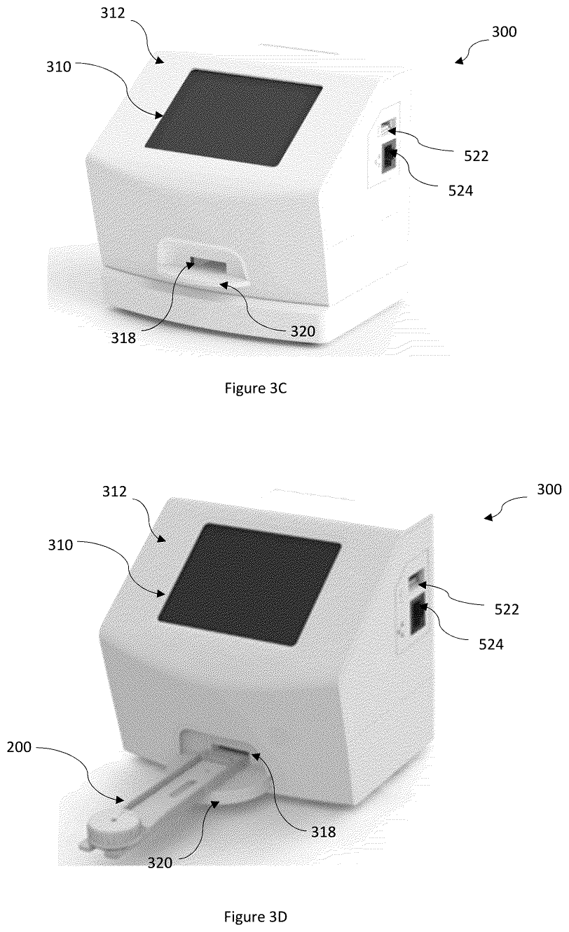

FIG. 3A is an image of a multi-modal diagnostic test reading apparatus (also referred to herein for brevity as a “multi-modal reader”) 300 according to an embodiment of the present invention, and which is capable of accepting and reading lateral flow strip cartridges or fluidic cartridges, for example a cartridge 150 containing strip 100, or a cartridge 200, as described above.

In this embodiment, the reader 300 comprises front and rear covers 312, 314 and a display 310. The display 310 may provide, for example, directives to a user for conducting a test, and display test results. In some embodiments, the display is touch sensitive for receiving user input; for example, the user may select the operation mode of the reader or enter patient data.

In some embodiments, the display 310 provides an overlayed or an otherwise combined visual representation of results obtained in multiple reading modes. The visual representation may include a virtual representation of the cartridge under test. The virtual cartridge may include visually readable test regions that represent non-visually readable regions of the cartridge under test.

The multi-modal diagnostic test reading apparatuses described herein comprise a diagnostic test assembly receiving component configured to receive a diagnostic test assembly. In the embodiment of FIG. 3A, this is in the form of a drawer 304 of reader 300 that allows a diagnostic test cartridge 150, 200 to be inserted into the reader 300. The drawer may be attached to the reader 300, or the drawer 304 may be removable. Placement of a cartridge 150, 200 into the drawer 304 may be detected by cartridge presence sensors 540 (not shown). Cartridge presence sensors 540 may comprise, for example, optical detectors or mechanical switches.

In some embodiments, the drawer 304 may be manually pushed into the reader 300. Alternatively, a drawer motor or actuator 530, for example a stepper motor, may automatically slide the drawer 304 into the reader 300 when, for example, the cartridge presence sensor 540 detects that the cartridge 150 is correctly inserted or upon instructions from a device controller 514 of the reader 300, as described below.

In some alternative embodiments, as shown in FIGS. 3C and 3D, the reader 300 does not have a drawer, and the cartridge 150, 200 is instead inserted directly into an opening 318 in the reader 300. It will be apparent to those skilled in the art that in other embodiments other arrangements may be used for inserting a cartridge into the reader 300, or otherwise aligning a cartridge with the device image sensor.

In the described embodiments, input/output ports are provided; for example, an Ethernet port 524 for connection to a PC or a server and/or a network printer, and one or more USB ports 522 for connecting to, for example a Seiko SPL620 or other label printer for printing test report receipts, or a USB flash memory key. However, in other embodiments, the reader may include a wireless network interface to allow the reader to wirelessly communicate with computers, printers and/or other networked devices.

FIG. 3B is a rear view of the reader 300 of FIG. 3A. The reader 300 includes a power connector 302, however in other embodiments, the reader 300 may be battery powered. Some further optional features include a speaker 306 for providing audible instructions and/or feedback to a user or other chimes/sounds for improved usability, an on/off switch 305, and an anti-theft security slot 308 for use with, for example, a Kingston security lock. In an alternative embodiment, the instrument may simply turn on when it is connected to an external power supply, in which case the on/off switch 305 is not required. Screws within screw holes 316 affix the front cover 312 to the back cover 314.

FIG. 3C is an image of an alternative embodiment of a multi-modal reader 300, wherein instead of a drawer 304, the reader 300 includes an opening 318 into which a cartridge 150, 200 is inserted. Similarly, the embodiment of a multi-modal reader 300 depicted in FIG. 3D has an opening 318 for cartridge insertion, and is depicted with a fluidic cartridge 200 positioned for insertion into the opening 318. The embodiments of FIG. 3C and FIG. 3D have stabilising platforms 320 to assist with cartridge insertion.

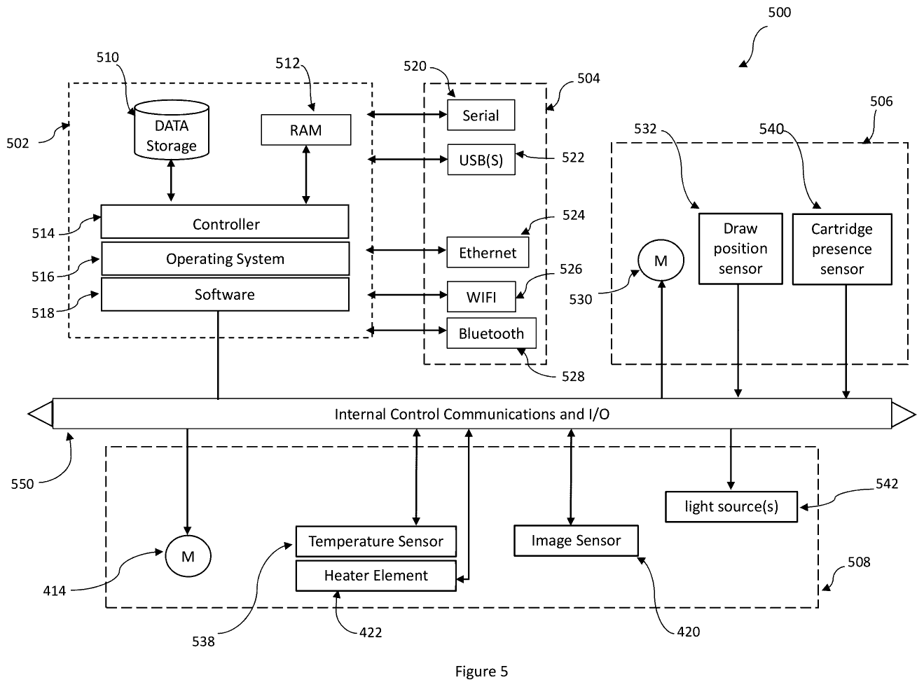

FIG. 5. is a block diagram of a control component 500 of a multi-modal reader 300 according to some embodiments of the present invention. The control component 500 includes a storage and processing component 502 comprising non-volatile data storage 510 for creating and maintaining, for example, a database of test results and relevant test information, operating random access memory (RAM) 512, a controller 514 that executes instructions of custom software 518 and an operating system 516, these being stored on the non-volatile data storage 510.

In the described embodiments, the multi-modal diagnostic test process executed by the multi-modal reader is implemented in the form of executable instructions of the custom software 518 executed by the controller 514. However, it will be apparent to those skilled in the art that at least a portion of the process can alternatively be implemented in one or more other forms, for example as configuration data of a field-programmable gate array (FPGA), or as one or more dedicated hardware components, such as application-specific integrated circuits (ASICs), or as any combination of such forms.

In some embodiments, the controller of the apparatus is a single-chip microcontroller that includes dynamic storage memory, non-volatile programmable memory for the arithmetic functions, and I/O interfaces. For example, in some embodiments the controller is an i.MX 8 Series multicore processor based on the Arm™ Coretex™ architecture and available from NXP Semiconductors™.

The external data interfaces 504 may include a serial port 520, one or more USB ports 522 for, for example, exporting data or connecting to other devices for example a printer, Ethernet 524 for data transfer, and in some embodiments includes WiFi 526 and Bluetooth 528 interfaces for cloud-based data storage and management, for example.

In some embodiments, real-time data obtained by the reader automatically populates a laboratory information system (LIS) or another database. This data may then be used to prompt immediate health responses. Alternatively, or additionally, the data may be linked to a stock control and procurement system.

A drawer control component 506 comprises drawer motor or actuator 530, which operates to open or close the drawer 304 upon receipt of instructions from the controller 514. Control of the drawer motor or actuator 530 may be dependent on information received from the drawer position sensor 532 and the cartridge presence sensor 540. In some embodiments, the motor or actuator 530 operates to open or close the drawer 304 based on input received from a user.

A reading control component 508 is also provided, and includes a filter motor or actuator 414, temperature sensor 538, heater element 422, image sensor 420 and one or more light sources 542. As described in more detail below, a stimulating signal/excitation or other light source 542 may be required to enable the image sensor 420 to read the test results. As the instrument is capable of operating in multiple reading modes, the one or more light sources 542 are selectively energised when required, by the controller 514.

If one or more of the tests being read requires temperature regulation, the reader 300 may include a heater element 422 located in close proximity to the cartridge under test. Temperature control is provided by way of a temperature feedback loop between a temperature sensor 538, the heater element 422, and the controller 514.

Further, depending on specific configuration and the reading modes provided by the reader, one of possibly multiple optical filters may be placed in front of the image sensor 420 in particular modes. Movement of the one or more filters is effected by the filter motor or actuator 414.

Communication between the storage and processing component 502, the drawer control component 506, and the reading control component 508 occurs by way of an internal control and communications bus 550.

The reader 300 may further include a GPS module (not shown), which enables geospatial data acquisition to be combined with diagnostic test results. In the case of a biomedical diagnostic test, for example, relating to a particular disease, combining test results and GPS information enables automated mapping, as well as predictive analytics or machine learning to determine disease emergence, geographic ‘hot spots’, spread, community transfer rate, transmissibility, etc. Further, different regions may have different regulatory requirements, and these requirements may be automatically reflected in the content of test result outputs, based on the test location.

Where patient data is entered into the reader 300, the above analysis may be further enhanced. For example, by combining test results, GPS-based environmental factors and patient data such as age, gender, ethnicity, diet, blood-type, pre-existing conditions, current treatments and medicaments, etc., vital insights may be obtained. These insights will be especially pertinent for new diseases, and may, for example, be used to identify measures for slowing the spread of a disease, protect vulnerable people and identify, for example, sources of immunity, possible treatments and areas where further research is needed.

### Image Acquisition

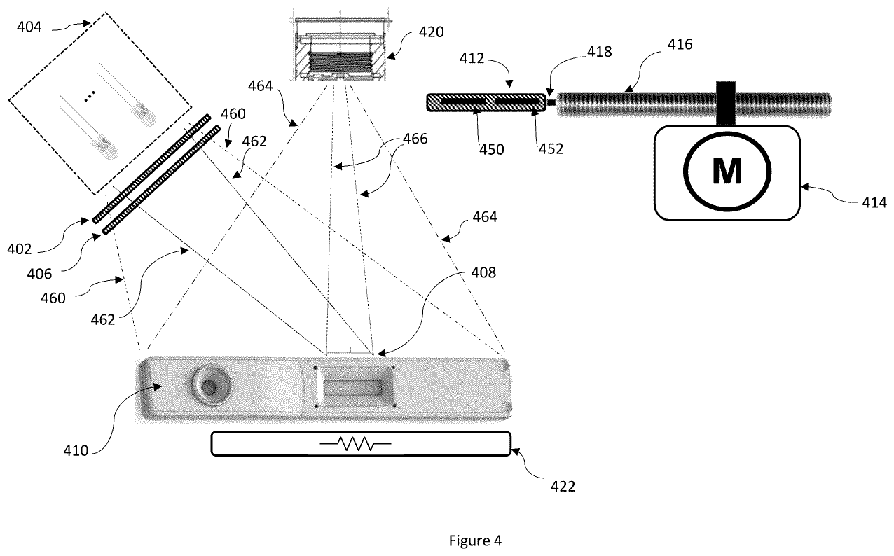

FIG. 4 illustrates the interaction between components of an example embodiment of a multi-modal reader 300. Here, the reader 300 is depicted as reading a cartridge 410, which may be a fluidic biological sample test cartridge, or a cartridge containing a lateral flow strip.

In the illustrated embodiment, a LED assembly including one or more light emitting diodes (LEDs) 404 may be used to illuminate/energise at least the test area 408 of the cartridge 410, as indicated by lines 462 in one reading mode, to enable an image sensor 420 to read test features as indicated by lines 466. The test area 408 may be, for example, the viewing window 240 or a region within the viewing window 240 as shown. Alternatively, the LEDs 404 may illuminate the whole cartridge 410, as indicated by lines 460, such that the reader 300 can read other cartridge features outside of the test area 408, as indicated by lines 464. For example, where the cartridge is similar to the cartridge 150 of FIG. 1B, the reader 300 can read the cartridge label 144 (if present) and the QR code 142 (if present).

For absorption/reflection (AR)-based reading of colorimetric tests, the best read is achieved when the peak wavelength of the illumination signal is the same or similar to the wavelength(s) of the visual feature(s) that indicate the test result. Conversely, for fluorescence-based readings the excitation source wavelength(s) is selected to be different, and non-overlapping with, the wavelength of the stimulated emission signal.

Multiple LEDs may be used to provide a source having a more even spatial distribution of illumination. In some embodiments, two sets of multiple LEDs are provided. One set of LEDs is for use in a first reader mode and its LEDs are all of the same type and have the same centre wavelength, and the other set of LEDs is for use in a second reading mode, and its LEDs are of the same type and have the same centre wavelength, where the centre wavelengths of the two sets of LEDs are different.

The LEDs may be mounted to the surface of a printed circuit board of the apparatus, wherein the controller 514 selectively energises the first set of LEDs when the device is in a first reading mode, and the second set of LEDs when the device is in a second reading mode.

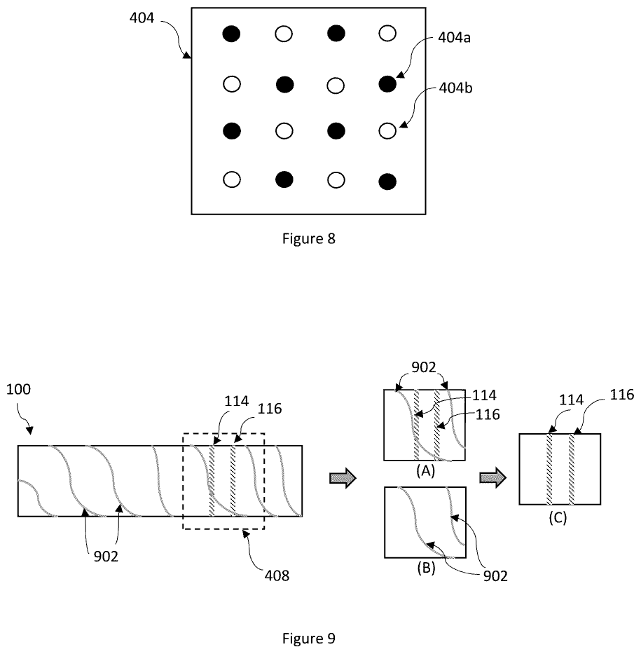

FIG. 8 is a schematic plan view of an example LED array for use in an embodiment of the present invention. The array comprises two sets of LEDs, wherein the black circles 404a represent LEDs of a first wavelength, in an energised or ‘on’ state. The white circles 404b represent LEDs with a second wavelength different from the first wavelength, and which are in a de-energised or ‘off’ state. As will be apparent to those skilled in the art, many alternative LED array arrangements can be used to achieve similar results. Further, in an alternative arrangement, LEDs of different sets may be mounted to different, moveable surfaces, such that only the light produced by a single set of LEDs is incident on the test area 408 or cartridge 410 at any time.

Returning now to FIG. 4, the LED assembly may additionally include one or more diffusers 402, 406 disposed between the LEDs 404 and the test area 408/cartridge 410 to further disperse and improve the evenness of the excitation/illumination of the cartridge 410.

In one or more reading modes, an optical filter, for example a bandpass filter, may be movably positioned between the test area 408 or cartridge 410 and the image sensor 420. In one embodiment, a movable slide 412 may comprise two filter positions 450, 452 into which optical filters may be inserted.

In a first reading mode, the controller 514 can instruct the motor or actuator 414 to position the slide 412 such that a first filter is positioned in the direct optical path between the image sensor 420 and the cartridge 410 under test. In a second reading mode, the second filter may be positioned between the image sensor 420 and the cartridge 410.

If, however an optical filter is not required for a particular reading mode, one of the slide positions 450, 452 may remain empty, or the slide 412 may simply be positioned out of the way of the image sensor 420.

Linear movement of the slide 412 is controlled by the motor or actuator 414. In some embodiments, the slide 412 is attached to the motor or actuator 414 by a lead screw 416 and nut 418. Alternatively, or additionally, the slide may be configured to move within a channel or guide rails (not shown).

If required, the reaction temperature for the lateral flow strip can be controlled using heating element 422, which may comprise, for example, one or more resistors. A temperature sensor 538 (not shown) provides feedback in a standard temperature control circuit. Electrical connections to the heating element and temperature sensor may be by way of cable connections or slip rings.

The image sensor 420 may be, for example, a 5-megapixel CMOS sensor, a CCD sensor or an arrangement of photodiodes. The image sensor 420 may further include an integrated red, blue, and green Bayer filter 700 or other colour filter to increase the detection sensitivity for visible wavelengths. FIG. 7 provides an example of a known Bayer filter arrangement 700, comprising an array of green 710, red 712 and blue 714 filters.

The wavelengths of LEDs 404a and 404b, and the optical wavelength filters for filter positions 450, 452 are selected to suit the different reading requirements of the specific test(s).

The multi-modal reader can be configured to change between the two or more reading modes in response to, for example, a user selection made using an apparatus user interface, or, for example, by automatic detection of the test cartridge type. The reader 300 may then operate according to a workflow suitable for the specific cartridge under test 410.

In various embodiments, the test cartridge type can be identified visually, by processing an initial image or images to determine one or more visual indicia or other visual features (e.g., shape) of the test cartridge, or alternatively by one or more non-imaging sensors determining a physical shape or physical feature of the test cartridge (e.g., by determining the actuation or non-actuation of one or more microswitches of the test apparatus receiving portion of the apparatus, or the blocking or non-blocking of one or more light beams), or by electronically reading or measuring an electronic feature or microchip of the test cartridge.

In each reading mode, the reader may obtain a single measurement, or the reader may obtain repeated measurements over time to produce a temporal kinetic readout. A temporal kinetic readout may be obtained to monitor, for example, changes in binding events over time or enzyme activity. A non-limiting example of a repeated measurement application is described in detail below in example configuration 3.

### Example Configuration 1

In a first example configuration, a multi-modal reader 300 is configured to read colorimetric test or control lines with colloidal gold/red labelled particles of a lateral flow strip in a first reading mode, and fluorescence-based immunoassay capture lines with europium chelate fluorescence labelled particles in a second reading mode.

In a first reading mode, an absorption/reflection (AR)-based image is captured. Green 520 nm LEDs are provided for narrow-band illumination of the test area 408, and the first position 450 of slide 412 is intentionally left empty, i.e. all wavelengths are allowed to pass. Here, a bandpass filter is not required to read the colorimetric immunoassay capture lines; however, in this embodiment the sensor 420 includes a Bayer filter.

In the first reading mode, upon instruction from the controller 514, green LEDs 404a are positioned and/or energised to illuminate the test area 408, and the controller 514 instructs the motor or actuator 414 to move the empty filter location 450 in front of the image sensor 420.

As a Bayer filter is used in this embodiment, an image captured in the first reading mode comprises pixels acquired with red, blue and green pixel filter elements. The controller 514 selectively uses only the green filter element pixels for the reading because these pixels have very high sensitivity to the green absorption at around 520 nm, which is the characteristic maximum absorption wavelength of colloidal gold particles.

Additionally, or alternatively, a green colour filter may be located or moved in front of the sensor 420. Using a narrower green bandpass filter of the same wavelength range as the incident light source, in combination with the green Bayer filtered pixels, can produce a more accurate AR-based reading.

For the second reading mode, the second filter position 452 of slide 412 is fitted with a 615 nm centred bandpass filter, and second reading mode LEDs 404b are selected to provide UV emissions with a centre wavelength of approximately 360 nm.

In the second reading mode, the controller 514 positions and/or energises the UV LEDs 404b to stimulate the test area 408. The filter position 452 is positioned in front of the sensor 420. This second reading mode arrangement provides an ideal configuration for the image sensor 420 to detect signals produced by the europium chelate fluorescence labelled particles.

In this way, the same reader 300 is capable of reading a lateral flow strip with test and/or control lines being either or both colorimetric and fluorescence labelled particles. The acquired images can then be used in subsequent image analyses.

The present configuration is described above with regard to test and control lines of a lateral flow strip. However, as will be appreciated, the same or a similar configuration may be also be used to read lateral flow strips with otherwise shaped test and control regions (e.g. dots), a fluidic cartridge or other type of test assembly, where the test involves colloidal gold and europium chelate fluorescence labelled particles.

Both readings may be, for example, displayed to a user via a user display 310. Results may alternatively or additionally be used by the controller 514 to produce a test result that combines data from both of the reading modes to form a result that could not be arrived at using individual reading modes only. Direct and calculated results may then be stored in memory 510.

### Example Configuration 2

In a second example configuration, a multi-modal reader 300 is configured to read immunoassay capture lines of a lateral flow strip with, for example, colorimetric immunoassay capture lines with colloidal gold particles in a first reading mode, and to detect one or more lateral flow strip artefacts, for example background staining, in a second reading mode. The multi-mode reader 300 can then remove or at least mitigate the effects of the artefacts on the test result.

Background staining can occur in lateral flow strips tests involving inherently coloured samples. Background staining is common, for example, in lateral flow strips designed to test for gastrointestinal related illnesses using diluted stool samples. As a sample progresses along the strip, visible staining may be produced. If only a single reading mode of imaging is used, the staining may interfere with interpretation of the test results.

For example, if one or more flow stain lines are formed in the proximity of the immunoassay capture lines, the staining may interfere with the determination of the test or control line values. For example, a qualitative test may produce a false positive result, or a quantitative test may produce overstated values.

In order to address such difficulties, one configuration of the multi-mode reader 300 corrects for background staining by capturing two or more images of the test region in two or more reading modes.

Unlike colloidal gold particles which have a narrow spectral response, stains have a broad spectral response, and the staining will therefore be present in images captured under a wide range of illumination and capture conditions. Image analysis, including subtracting one image from another image, can provide a combined result that removes or at least alleviates the impact of the staining on the test results.

In a first reading mode, the multi-mode reader 300 captures an absorption/reflection (AR)-based image, which is particularly sensitive to the colloidal gold particles of the immunoassay capture lines. A similar LED and filter arrangement may be used as described above with regard to example configuration 1. Due to the broad spectral response of the stain, the first image will include both the capture lines, if present, and some background staining.

In a second reading mode, a second AR-based reading mode is used to obtain an image of the staining only, wherein the reading mode has low sensitivity to the colloidal gold particles, but is approximately equally sensitive to the background staining.

For example, in a second reading mode, red LEDs may be selected, with a wavelength within the range of 620 nm-660 nm. For example, LEDs of wavelength 640 nm may be used to illuminate the test area 408. In the second reading mode, the controller 514 selectively uses red Bayer filter element pixels for the reading. These red filtered pixels have low sensitivity to the emissions from the colloidal gold particles. Therefore, an image captured in the second reading mode will include the staining only. In an alternative arrangement, rather than using a Bayer filter, a red colour filter may be located or moved in front of the sensor 420.

A staining scenario is depicted in FIG. 9, with a lateral flow strip 100, including, for example, visible colloidal gold test and control lines 114, 116, and staining 902. A first image (A) of the test area 408 is captured in the first reading mode of this embodiment, and a second image (B) of test area 408 is captured in the second reading mode of this embodiment. Once both images have been captured, the controller 514 may then subtract image (B) from image (A) to generate a new, calculated or ‘synthetic’ image (C).

Image (C) is free from, or at least less affected by, the staining 902, and can therefore be used to more accurately determine the presence or absence of the control and test lines 114, 116, and/or be used for increasing the accuracy of quantification.

As will be appreciated, other similar undesirable lateral flow strip artefacts can be subtracted from the detected image using a similar method, for example dirt, dust, or imperfections of the strip itself. Further, while the present example configuration is described above with respect to test and control lines, the same or a similar configuration can also be used to reduce the effects of lateral flow strips with otherwise shaped test and control regions (e.g., dots). A similar method may be used to reduce the effects of dirt, dust, reflections etc. in images received from a fluidic cartridge 200.

### Example Configuration 3

In a third example configuration, a multi-modal reader 300 is configured to obtain fluorescence-based readings relating to accumulating concentrations of NADPH in a fluidic cartridge 200 in a first reading mode, and absorbance-based haemoglobin (Hb) measurements in a fluidic cartridge 200 in a second reading mode. The rate of change of NADPH measurements overtime, which can be determined from two or more images, provides an indication of the amount of glucose-6-phosphate dehydrogenase (G6PD) enzyme present in a sample. Combined knowledge of G6PD and Hb levels in a sample is relevant for informing treatment decisions when treating humans with malaria.

Presently, drugs are available for treating latent malaria infection; however, these drugs are potentially harmful if administered to patients with low levels of the enzyme G6PD relative to Hb. Quantifying G6PD activity requires a compensation process that also accounts for a quantified Hb level. Therefore, multiple reading mode outputs are required to determine an appropriate treatment for a patient.

A cost effective G6PD test is therefore pertinent for the safe treatment of malaria, wherein the test is capable of determining the patient's G6PD level, Hb level, and then calculating, for example, a ratio of G6PD activity to Hb level. This ratio of G6PD enzyme activity to Hb level, also referred to as the “compensated G6PD value”, can then be used by clinicians to determine potential risks of drug applications or treatments.

G6PD is active in essentially all types of cells, and is involved in the normal processing of carbohydrates. It is responsible for the first step in the pentose phosphate pathway, a series of chemical reactions which includes converting the oxidised form of NADP, referred to as NADP⁺, to NADPH. The rate at which this conversion occurs is known to be a measure of the amount of G6PD present. The global health organisation PATH has produced a commonly used guide to fluorescent spot testing for G6PD deficiency.

The multi-modal reader of the present embodiment may be used to provide a diagnostic test result that combines NADPH test results and Hb levels, wherein the example fluidic cartridge 200 of FIGS. 2A and 2B may be used to perform the test.

In an example test configuration, a 5 μl blood sample and 500 μl of sample buffer solution is used in the test. As the person skilled in the art will appreciate however, many different volumic arrangement may be used in alternative test configurations.

The blood sample may be acquired from the patient using a finger prick with a sterile lancet, and transferred from the finger droplet to the test cartridge 200 using either a transfer device or by directly applying the finger droplet to a feature of the cartridge 200. The sample preparation buffer liquid is preloaded in the sample chamber 208 of cartridge 200. Further, the sample preparation buffer fluid can be an aqueous solution with detergent, salt, hypertonic water, or other reagent configured to cause the lysis of red blood cells within the added samples and to distribute the haemoglobin throughout the solution. Further, the reaction chamber 202 is preloaded with a soluble, dried or lyophilised reagent containing NADP⁺.

To start the test, the sample is added to the sample chamber 208, and the dispense cap 210 is screwed on. The cap 210 may include a piercing tip, which pierces a seal 206, allowing the blood sample, diluted with the aqueous solution, to flow into the reaction chamber 202. Once inside the reaction chamber 202, the G6PD present in the sample starts to convert the preloaded NADP into NADPH.

The NADPH generated by this reaction is a naturally fluorescent molecule, with a fluorescence wavelength around 500 nm. In the first reading mode, stimulating LEDs 404a may be selected to illuminate the viewing window 240 with a UV wavelength in the range of 320 nm to 380 nm. For example, LEDs 404a may be selected with a centre wavelength of approximately 350 nm. Further, for the first reading mode, a band pass filter with a non-overlapping (the stimulating UV wavelength) pass band may be placed in the first filter location of slide 412, such that, in the first reading mode, the excitation signal of the NADPH alone is detected by the image sensor 420.

The measured level of fluorescence is proportional to the quantity of NADPH present in the reaction chamber 202 at the time the fluorescence response image is acquired. A set of NADPH levels can be measured over time, wherein the slope/gradient of the NADPH increase over time, i.e. the rate of change in the NADPH level, can be used to calculate the relative level of G6PD present in the sample. That is, the more G6PD present, the faster the preloaded NADP will be converted to NADPH. Therefore, in a first reading mode, multiple NADPH measurements are made using sensor 420, and the controller 514 processes these measurements to calculate the amount of G6PD within the sample.

The above reaction is temperature dependent, and therefore the temperature of the cartridge, and more specifically, the temperature of the reaction chamber 202, may be maintained at a specific known temperature for the duration of the test. For example, the temperature for the reaction may be selected to be between 38° C. and 45° C. More specifically, the temperature may be maintained at 40° C. throughout. To maintain the temperature, the reaction chamber 202 may be in contact with or in close proximity to, for example, an anodised aluminium or ceramic block (i.e., to increase thermal mass) attached to the heating element 422.

Alternatively, the temperature of the cartridge 200 may not be controlled, but rather simply measured throughout the reaction. In this case, the controller 514 uses temperature measurements to adjust the NADPH rate calculation, in order to determine the level of G6PD.

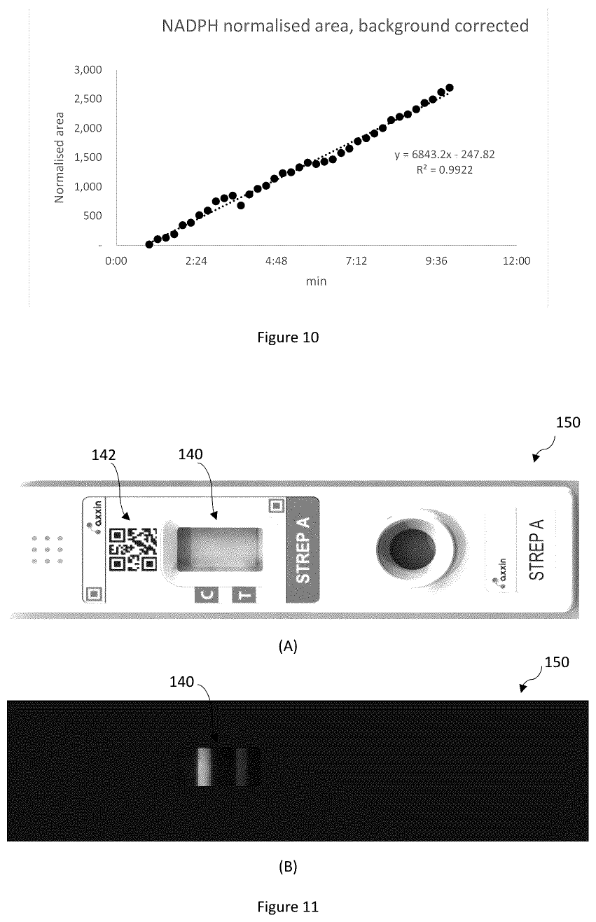

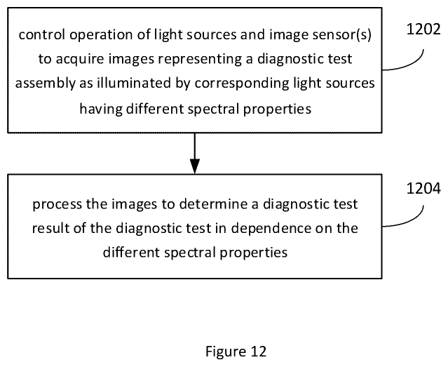

FIG. 10 provides an example NADPH versus time graph, wherein NADPH readings were obtained by a multi-modal reader according to an embodiment of the present invention, wherein the temperature of the reaction was constant. The test process used by the reader to determine the level of G6PD from the measurements may be based on a built-in test library, initially created using hundreds of clinical samples. Alternatively, in another embodiment, the reader may be calibrated by the user, using a range of samples with known concentrations of the G6PD enzyme.

In a second reading mode, the reader obtains a Hb measurement. Blue LEDs are used to illuminate the viewing window 240 of the cartridge 200 under test, and the image sensor 420 comprises a Bayer filter. The controller 514 selectively uses only pixels acquired with blue filter elements. Additionally or alternatively, a blue filter may be placed in a second filter position 452 of slide 412. The resulting image or digital representation of the viewing window 240 is then analysed to determine the amount of Hb present in the sample. Hb, being red, also absorbs green light well, and therefore, in an alternative embodiment, green illumination with appropriate green filtering may be used.

Once both the G6PD and Hb levels in the sample have been determined by the controller 514, the controller 514 then combines measurements obtained from the first and second reading modes to calculate the compensated G6PD value. This value can then be communicated to the user, for example via the display 310.

As will be appreciated by the skilled person, G6PD results are not only relevant when treating latent malaria. A wide range of applications exist for a point-of-care reader able to determine G6PD levels. A multi-modal reader as described herein may, for example, be configured to read test cartridges in one reading mode and other either related and/or unrelated test cartridges in further modes. Alternatively, a point-of-care reader as described herein may be configured to use multiple reading modes to read any other desirable multiplexed G6PD test.

Similar repeated measurement methods may be used to obtain other temporal kinetic readouts for monitoring, for example, changes in binding events over time, or the activity of other enzymes, which may then be combined with results or information obtained in other reading modes.

### Example Configuration 4

When performing any diagnostic test, it is critically important to ensure that the test results are reliably linked to the corresponding patient. Test cartridges may therefore have identifying marks such as a code 39, an EAN or PDF-417 barcode, a 2D Data Matrix, or a QR Code 142 printed or otherwise marked onto the surface of the cartridge. Further, the identifying mark may include encoded information about the test or cartridge itself, which reading mode or sequence should be used for reading and interpreting the test results, and any relevant calibration information.

In some embodiments, an external scanner may be used to read the identifying mark. For example, reader 300 may be configured to accept test specific inputs from an external scanner, for example user ID and test ID. An external reader may be connected via, for example, a USB port 522. The external scanner may be, for example, a Datalogic QuickScan™ Wand QD2430, which is capable of reading a variety of identifying marks. In the present embodiment however, the multi-modal reader 300 itself is configured to detect and interpret any such identifying marks.

For cartridge/sample storage/retrieval/identification/management (i.e., for purposes other than determining diagnostic test results), identifying marks should be visually readable by standard readers (e.g., a standard barcode reader) separate to the multi-modal readers described herein. Standard barcodes will not be available in images produced by readers configured to read only fluorescence-based tests. Indeed, prior art fluorescence readers cannot read barcodes. This embodiment overcomes this limitation by providing both absorption/reflection (AR) and fluorescence-based reading modes.

Further, if an identifying mark does not identify the cartridge type, then the reader 300 may use an AR-based reading mode to obtain further cartridge information. For example, the reader 300 may determine the outline of the cartridge for comparison with a database of known cartridge shape and feature dimensions. Further still, alternatively, or additionally, the reader 300 may use an AR-based reading mode to locate the position of the viewing window 140, 240.

Turning now to FIG. 11, FIG. 11A depicts an image of a cartridge 150 captured in an AR-based reading mode, and FIG. 11B depicts an image of a cartridge 150 captured in a fluorescence-based reading mode. As is apparent in FIG. 11B, cartridge features are not apparent in the fluorescence-based reading mode.

In FIG. 2B, two immunoassay capture lines are apparent in the fluorescence-based image, i.e. a test and control line. However, where only a single line is present, it is critically important to know the relative positioning of the line with respect to the viewing window 140, i.e. whether the single line is a test or control line. For example, for a qualitative diagnostic test, if only a control line is present, the test result is negative, however if only a test line is present, an error has occurred, and a result cannot be determined.

In the present embodiment, a first, AR-based, reading mode is provided, such that the instrument can read features of a cartridge 150, 200. A standard edge detection method is used to determine the coordinates of edges of the viewing window 140. Advantageously, in a multi-modal reader, the cartridge does not move between image captures, and coordinates of the viewing window may be used when interpreting an image captured in a second or any further subsequent reading modes.

For the first reading mode, a visible image can be obtained by the image sensor using red LED illumination of a selected wavelength within the range of 620 nm-660 nm, for example, red LEDs of wavelength 640 nm, to illuminate the cartridge features. Either red Bayer filter elements may be used for the reading, or alternatively a red filter may be placed in a second filter position 452 of slide 412 and be positioned in front of the sensor 420.

In the first reading mode, reflected light from the cartridge features passes through the red band pass filter with the same band pass filter for detection in the image sensor. This reading mode obtains an image of the data code and/or features of the cartridge. Image analysis by the controller 514 can then extract data from the image, for example the encoded information from a data code and/or viewing window coordinates. The data code may include information such as calibration factors and expiry dates, which are then available for combining with results of other reading modes to generate a final test result. For example, the expiry date may indicate whether or not accelerated product degradation is likely to have occurred, and incorporating an expiry date into the diagnostic test process may therefore lead to a more accurate result.

For the second reading mode, the reader 300 may be configured to obtain, for example, a fluorescence-based reading in accordance with any of the above described fluorescence reading modes, or any other desirable reading.

In a specific example, if a Europium fluorescence-based test is used in the cartridge 150, 200, then a fluorescence image can be produced using UV illumination. The same red bandpass sensor filter can be used in the second reading mode, as in the first, AR-based reading mode. This specific configuration of reading modes has the advantage that the image sensor band pass filter is not required to move/change position between reading modes.

### Example Configuration 5

In a fifth example configuration, a multi-modal reader is configured to obtain absorption/reflection (AR)-based readings relating to sample flow in a fluidic cartridge 200 or along a lateral flow strip 100 in a first reading mode, and fluorescence-based measurements in second reading mode.

As described above, lateral flow strips often include a control immunoassay capture line to indicate that the sample has successfully migrated across the strip. However, in some configurations, including tests involving a fluidic cartridge, a control measure may be desirable as an alternative or in addition to other controls. If the flow can be observed in an AR-based reading mode, i.e. the materials are not fluorescent, then information relating to the flow can be used in the test process. Fluorescence can then be independently measured.

For tests involving blood, for example, sample flow through a cartridge or lateral flow strip may be inherently capable of being imaged using an AR-based reading mode. In other samples, for example urine or a dilute sample, a visual dye may be added, such that flow is evident. Alternatively, the flow may be evident as discolouration due to wetting.

The presence of the flow, including its completion through the full length of the strip, or into/through a reservoir/channel/chamber etc. of a fluidic cartridge can be utilised within the test process as an improved control to confirm that the sample was adequately added to the test area 408.

Additional characteristics of the sample flow reading mode, such as the rate of progress of the leading edge of the flow or an AR-read quantification measurement may be used to determine the amount of sample added, or at least to confirm that an adequate amount of sample was added. The controller 514 may then use the multiple readings to perform calculations that allow, for example, for the amount of the analyte detected to be compensated for by the amount of sample applied. That is, an optical AR-based measurement can be used to detect the quantity/amount of a sample material applied to the test strip of a test cartridge, and fluorescence detection can be used to detect the level of a specific analyte within the sample.

The controller 514 can then combine the multiple measurements to generate an overall test result. For example, the reading may prevent an incorrect test result from being generated if an inadequate amount of sample was added.

In an example embodiment, a blue dye may be added to the sample, which has, for example, absorption in the range of 440-490 nm, wherein the dye is visually detectable in a first, AR-based reading mode.

In a second reading mode, UV LEDs in the range 340-370 nm may excite, for example, Europium fluorescence of a test region. The image sensor 420, with an appropriate bandpass filter, obtains a reading of the resulting red emission within the wavelength range 605-625 nm. As the sample is stained with a blue dye, the dye does not interfere with the fluorescence measurement in the fluorescent reading mode.

### Other Embodiments

The above example embodiments involve immunoassays and enzymatic assays; however, as those skilled in the art will appreciate, the multi-modal readers described herein may also be used to perform analogous diagnostic tests involving nucleic acid amplification assays.

Further, in any embodiment of the invention where the reader is configured to read visually interpretable cartridges or test strips, the reader may be further configured such that a user's subjective visual interpretation of a test may be entered as input into the device. The controller 514 may then correlate the user's subjective interpretation with an objective test result or other analysis performed by the reader, which can advantageously be used to determine end-user competencies and identify user training requirements.

While the above example embodiments generally describe using different reading modes to read different types of tests, and/or different cartridge or test area features, the multiple reading modes can also be used to gain further insights regarding the same test. Obtaining different absorption/reflection (AR)-based readings can be used to determine characteristics of a signal produced at a single test region. For example, if a lateral flow strip comprises a colloidal gold (red) colorimetric test line, it would be expected that reading modes that use green illumination and green filtering will produce the strongest AR-based reading. As a control measure, the signal can be checked by obtaining a further reading of the same immunoassay capture line using blue illumination and blue filtering or red illumination and red filtering. As will be appreciated, characteristics of both colorimetric and fluorescent test and control signals may be determined or verified using multiple readings with different optical filters.

Further, while the readers described herein were developed for the primary purpose of reading a diagnostic test assembly in multiple reading modes to determine a diagnostic result, some types of diagnostic test assembly may require only one reading mode, and the readers described herein can also be used with such diagnostic test assemblies.

### Incorporating Further Reading Modes

The multi-modal readers described above include two sets of LEDs 404a, 404b and two filter positions 450, 452; however, a person skilled in the art will appreciate that the readers can easily be adapted to include further reading modes. As will be apparent to the person skilled in the art, many combinations of the above described reading modes are possible, and the reader 300 may read in 2, 3, 4, 5 . . . 10 or more different modes.

To enable the further reading modes, additional sets of LEDs may be selectively energised or positioned to illuminate/energise a cartridge under test, and the slide 412 may be configured to accommodate more than two filters.

Multi-mode readers with a large number of optical filters may require an alternative arrangement for filter positioning. For example, the filters may be inserted into a rotary carousel, wherein the active filter is positioned between the test area 408/cartridge 150, 200 and the image sensor 420 by a motor rotating the carousel.

Alternatively, a variable filter may be used; for example, a Delata 3G LVSWP linear variable filter may be used. Instead of, or additionally to using multiple filters, a variable filter may be moved by motor or actuator 414, to provide a large number of reading modes covering a full spectrum. This may be used in addition to fluorescence-based reading modes.

Other mechanical arrangements for both the LED illumination of the test area 408 or cartridge and the active filter positioning will be apparent to those skilled in the art.

As used herein, except where the context requires otherwise the term ‘comprise’ and variations of the term, such as ‘comprising’, ‘comprises’ and ‘comprised’, are not intended to exclude other additives, components, integers or steps.

The foregoing summary is not intended to summarise each potential embodiment or every aspect of the present disclosure.

Many modifications within the scope of the present invention will be apparent to those skilled in the art in light of this disclosure.

1. A multi-modal diagnostic test reading apparatus, comprising:
a diagnostic test assembly receiving component;
at least one image sensor;
a plurality of light sources having respective different spectral properties; and
a controller;
wherein the controller is configured to:
(i) control operation of the light sources and the at least one image sensor to acquire a plurality of images, each of the acquired images representing at least a corresponding portion of the diagnostic test assembly as illuminated by a corresponding one of the light sources; and
(ii) process the acquired images to determine a diagnostic test result of the diagnostic test, the diagnostic test result being dependent upon the processed images representing illumination by respective ones of the light sources having respective different spectral properties.

2. The apparatus of claim 1, further comprising an optical filtering component operable by the controller to selectably locate a corresponding optical filter of one or more optical filters between the image sensor and the diagnostic test assembly to filter corresponding wavelengths from the image sensor when acquiring one or more corresponding images of the acquired images.

3. The apparatus of claim 1, wherein the controller is configured to include an operating mode wherein the plurality of images include:
an absorption/reflection-based image of a first colorimetric signal produced at a first test region of the diagnostic test assembly; and
an absorption/reflection-based image of a second colorimetric signal produced at a second test region of the diagnostic test assembly;
wherein the spectral properties of the first colorimetric signal are different to the spectral properties of the second colorimetric signal.

4. The apparatus of claim 1, wherein the image sensor includes a Bayer filter, and while acquiring respective images of the plurality of images, the controller is configured to selectively use only respective different subsets of pixels of the image sensor selected from three subsets of pixels of the image sensor with red, blue and green Bayer filter elements, respectively.

5. The apparatus of claim 1, wherein the controller is configured to include an operating mode wherein the plurality of images include:
an absorption/reflection-based image of a colorimetric signal produced at a first test region of the diagnostic test assembly; and
a fluorescence-based image of a fluorescent signal produced at a second test region of the diagnostic test assembly.

6. The apparatus of claim 5, wherein:
the colorimetric signal is a signal produced by colloidal gold labelled particles; and
the fluorescent signal is a signal produced by europium chelate fluorescence labelled particles.

7. The apparatus of claim 3, wherein the first and second test regions are first and second immunoassay capture lines of a lateral flow strip.

8. The apparatus of claim 1, wherein the controller is configured to include an operating mode wherein the plurality of images include:
an absorption/reflection-based image of a modified sample contained within a diagnostic test assembly, relating to a first property of the sample; and
multiple fluorescence-based images of a fluorescent signal produced by the modified sample relating to a second property of the sample.

9. The apparatus of claim 8, wherein:
the modified sample is blood mixed with a buffer solution;
the first sample property is an amount of haemoglobin in the sample;
the second sample property is an amount of nicotinamide adenine dinucleotide phosphate hydrogen (NADPH) produced while running the test;
wherein the controller is configured to determine, based on changes in the multiple fluorescence-based images over time, an amount of glucose-6-phosphate dehydrogenase (G6PD) present in the sample; and
wherein determining the test result comprises calculating the amount of G6PD relative to the amount of haemoglobin in the sample.

10. The apparatus of claim 1, wherein the controller is configured to include an operating mode wherein the plurality of images include:
a first, absorption/reflection-based image of one or more visual features within a test area of the diagnostic test assembly; and
a second image of at least a signal produced at a test region of the diagnostic test assembly, the second image being an image of either:
a fluorescent signal; or
a colorimetric signal;
wherein the diagnostic test result is dependent upon the one or more visual features and the signal of the processed images.

11. The apparatus of claim 10, wherein the one or more visual features comprise one or more of:
flow of a coloured sample through a fluidic test cartridge;
flow of a coloured sample along a lateral flow strip;
wetting of a lateral flow strip due to flow of a transparent sample;
variation in illumination;
background staining on a lateral flow strip;
dirt, dust, or imperfections of a lateral flow strip; and
reflections from a reflective surface of the viewing window.

12. The apparatus of claim 11 wherein:
the one or more visual features comprise background staining and variation in illumination level; and
the controller is configured to subtract the first image from the second image to produce a third image with reduced contribution from the background staining or variation in illumination level,
wherein the third image is used to determine the diagnostic test result.

13. The apparatus of claim 1, wherein the controller is configured to include an operating mode wherein the plurality of images include one or more absorption/reflection-based images of one or more features of the diagnostic test assembly, the diagnostic test result being dependent upon the one or more visual features, and wherein the one or more features comprise one or more of:
the outline of the diagnostic test assembly;
a viewing window of the diagnostic test assembly;
a data code printed or etched on the diagnostic test assembly; and
a label of or affixed to the diagnostic test assembly.

14. The apparatus of claim 13, wherein:
the one or more features comprise the data code,
the controller is configured to obtain information from the data code; and
the controller processes the information obtained from the data code to determine parameters including a test identifier, wherein one or more of the parameters are used to determine the diagnostic test result, and are displayed together with the diagnostic test result.

15. The apparatus of claim 1, further comprising one or more optical diffusers positioned between one or more of the light sources and a diagnostic test assembly received in the apparatus to improve illumination of the diagnostic test assembly.

16. The apparatus of claim 1, wherein the controller is configured to automatically determine one or more operating modes for acquiring the plurality of images, and to control operation of the light sources and the at least one image sensor in accordance with the determined one or more operating modes to acquire the plurality of images.

17. The apparatus of claim 1, wherein the controller is configured:
(a) to control operation of the light sources and the at least one image sensor to acquire at least one image of the plurality of images;
(b) to process the at least one image to determine one or more operating modes for acquiring one or more other images of the plurality of images; and
(c) to control operation of the light sources and the at least one image sensor in accordance with the determined one or more operating modes to acquire the one or more other images of the plurality of images.

18. The apparatus of claim 17, wherein the at least one image includes at least one absorption/reflection-based image of one or more of:
an outline of the diagnostic test assembly;
a data code printed on or etched into the diagnostic test assembly; and
a label of or affixed to the diagnostic test assembly;
wherein the controller is configured to determine an operating mode for acquiring at least one of the one or more other image by processing the at least one image to determine at least one of:
a type of the diagnostic test assembly represented in the image; and
a type of the diagnostic test of the diagnostic test assembly.

19. A process executed by at least one processor of a multi-modal diagnostic test reading apparatus comprising at least one image sensor and a plurality of light sources having respective different spectral properties, the process comprising the steps of:
(i) controlling operation of the light sources and the at least one image sensor to acquire a plurality of images, each of the acquired images representing at least a corresponding portion of the diagnostic test assembly as illuminated by a corresponding one of the light sources; and
(ii) processing the acquired images to determine a diagnostic test result of the diagnostic test, the diagnostic test result being dependent upon the processed images representing illumination by respective ones of the light sources having respective different spectral properties.

20. The process of claim 19, including a step of automatically determining one or more operating modes for acquiring the plurality of images; wherein the step of controlling comprises controlling operation of the light sources and the at least one image sensor in accordance with the determined one or more operating modes to acquire the plurality of images.

21. The process of claim 19, wherein the step of controlling comprises:
(a) controlling operation of the light sources and the at least one image sensor to acquire at least one image of the plurality of images;
(b) processing the at least one image to determine one or more operating modes for acquiring one or more other images of the plurality of images; and
(c) controlling operation of the light sources and the at least one image sensor in accordance with the determined one or more operating modes to acquire the one or more other images of the plurality of images.

22. At least one computer-readable storage medium having stored thereon processor-executable instructions and/or FPGA configuration data that, when executed by at least one processor of a multi-modal diagnostic test reading apparatus, and/or when used to configure an FPGA of a multi-modal diagnostic test reading apparatus, cause the at least one processor and/or the FPGA to execute the process of claim 19.
